(19) United States
(12) Patent Application Publication
(10) Pub. No.: US 2018/0235911 A1
(43) Pub. Date: Aug. 23, 2018

(54) STABLE PHARMACEUTICAL COMPOSITION OF ALOGLIPTIN AND METFORMIN FIXED DOSE COMBINATION

(71) Applicant: ALEMBIC PHARMACEUTICALS LIMITED, Vadodara (IN)

(72) Inventors: Dr. Pankaj KHAPRA, Vadodara (IN); Hitesh Vaikunthbhai THAKKAR, Vadodara (IN); Subhajit Chandra KAR, Vadodara (IN)

(73) Assignee: ALEMBIC PHARMACEUTICALS LIMITED, Vadodara (IN)

(21) Appl. No.: 15/753,691

(22) PCT Filed: Aug. 20, 2016
(86) PCT No.: PCT/IB2016/054993

§ 371 Date: Feb. 20, 2018

(30) Foreign Application Priority Data
Aug. 21, 2015 (IN) 3194/MUM/2015

(51) Int. Cl. A61K 31/155; A61K 9/16

(52) U.S. Cl. CPC A61K 31/155; A61K 9/1623; A61K 9/1635; A61K 31/513; A61K 9/1682; A61K 2300/00; A61K 9/1652

(57) ABSTRACT

The present invention relates to a solid oral preparations comprising alogliptin and metformin fixed dose combination and to a process for preparation thereof. More particularly, it relates to a solid oral preparation of alogliptin and metformin fixed dose combination formulation which is stable and easy to manufacture.

### TECHNICAL FIELD

The present invention relates to a solid oral pharmaceutical composition comprising alogliptin and metformin fixed dose combination and a process for preparation thereof. More particularly, it relates to a solid oral pharmaceutical composition of alogliptin and metformin fixed dose combination wherein the said fixed dose combination is stable.

### BACKGROUND AND PRIOR ART

Alogliptin, 2-({6-[(3R)-3-aminopiperidin-1-yl]-3-methyl-2,4-dioxo-3,4-dihydro-2H-pyrimidin-1-yl}methyl) benzonitrile, is an orally administered anti-diabetic drug. The structural formula is as below:

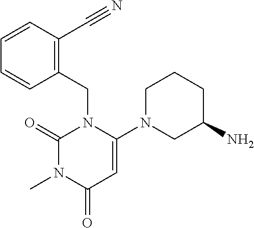 [SMILES: Cn1c(=O)cc(N2CCC[C@@H](N)C2)n(Cc2ccccc2C#N)c1=O]

Alogliptin disclosed in US 2005/261271, EP 1586571 and in WO 2005/095381. WO 2007/035629, disclosed alogliptin is in the form of its benzoate salt, its hydrochloride salt or its tosylate salt. Polymorphs of alogliptin benzoate are disclosed in WO 2007/035372. A process for preparing alogliptin is disclosed in WO 2007/112368 and, WO 2007/035629.

The drug to be combined with the alogliptin benzoate within the pharmaceutical compositions is metformin such as metformin hydrochloride.

The biguanide antihyperglycemic agent metformin disclosed in U.S. Pat. No. 3,174,901. The preparation of metformin (dimethyldiguanide) and its hydrochloride salt is state of the art and was disclosed first by Emil A. Werner and James Bell, J. Chem. Soc. 121, 1922, 1790-1794. Other pharmaceutically acceptable salts of metformin can be found in U.S. application Ser. No. 09/262,526 filed Mar. 4, 1999 or U.S. Pat. No. 3,174,901.

It was observed that the fixed dose combination of alogliptin metformin was not chemically stable as primary and tertiary amino group of alogliptin show incompatibilities, degradation problems, or extraction problems with the excipients such as lactose and other reducing sugars. Though alogliptin itself very stable, but when it reacts with incompatible drug such as metformin, or its impurity product, and/or with many excipients used in solid dosage forms and with impurities of excipients, especially in tight contact provided in tablets and at high excipient/drug ratios. The amino group appears to react with reducing sugars. These unforeseen difficulties are primarily observed in low dosage ranges of the alogliptin, and/or high dosage ranges of the partner drug, like metformin.

U.S. Pat. No. 8,900,638 disclose stability related concerns with the fixed dose combinations of alogliptin and metformin when alogliptin was combined with metformin.

U.S. Pat. No. 8,900,638 disclose that in a compatibility test of alogliptin or a salt thereof, and metformin hydrochloride, no problem occurred. When a solid preparation comprising alogliptin or a salt thereof, and metformin hydrochloride was actually produced and subjected to a preservation stability test, a related substance of alogliptin (decomposed product derived from alogliptin) was detected. In other words, a decrease in the preservation stability of alogliptin or a salt thereof in a solid preparation was confirmed. For a preparation comprising alogliptin or a salt thereof, and metformin hydrochloride as active ingredients to be provided, therefore, it is necessary to prevent a decrease in the preservation stability of alogliptin or a salt thereof in the preparation.

U.S. Pat. No. 8,900,638 further discloses intensive studies in an attempt to solve the above-mentioned problems and found that a solid preparation comprising alogliptin or a salt thereof, and metformin hydrochloride, which are physically separated therein from each other, is superior in the preservation stability. U.S. Pat. No. 8,900,638 discloses a solid preparation comprising alogliptin or a salt thereof, and metformin hydrochloride, wherein the alogliptin or a salt thereof is physically separated from metformin hydrochloride.

US 20110206766 discloses certain stability related concerns with the fixed dose combinations of DPP IV inhibitors like sitagliptin, saxagliptin, vildagliptin and/or alogliptin and metformin when alogliptin was combined metformin.

US20110206766 further discloses that in attempts to prepare pharmaceutical compositions of selected DPP-4 inhibitors it has been observed, that the DPP-4 inhibitors with a primary or secondary amino group show incompatibilities, degradation problems, or extraction problems with a number of customary excipients such as microcrystalline cellulose, sodium starch glycolate, croscarmellose sodium, tartaric acid, citric acid, glucose, fructose, saccharose, lactose, maltodextrines. Though the compounds themselves are very stable, they react with incompatible partner drug, or its impurity product, and/or with many excipients used in solid dosage forms and with impurities of excipients, especially in tight contact provided in tablets and at high excipient/drug ratios. The amino group appears to react with reducing sugars and with other reactive carbonyl groups and with carboxylic acid functional groups formed for example at the surface of microcrystalline cellulose by oxidation. These difficulties are primarily observed in low dosage ranges of the DPP-4 inhibitor used, which are required due to their surprising potency, and/or high dosage ranges of the partner drug used. Thus, pharmaceutical compositions are required to solve these technical problems, which may be associated with the unexpected potency of selected DPP-4 inhibitor compounds.

It was also disclosed in US20110206766 that by the use of a nucleophilic and/or basic agent, which may be suitable for stabilizing, such as e.g. a suitable buffering agent as stabilizer, within these pharmaceutical compositions one can overcome these problems, e.g. of incompatibility and poor stability, especially decomposition and/or “assay decrease” which may be caused e.g. by reaction (e.g. by acylation, urea formation or Maillard reaction, or the like) of free base type DPP-4 inhibitors when combined with an incompatible partner drug, or its impurity product and/or a pharmaceutical excipient having such functional group (such as a reducing end of a sugar or an acyl group, such as e.g. an acetyl or carbamoyl group) to form derivatives with the free base type DPP-4 inhibitors, such as e.g. N-acetyl or N-carbamoyl derivatives. Therefore, by the use of a suitable nucleophilic and/or basic agent (e.g. a buffering and/or pH modifying agent) within these pharmaceutical compositions protection against decomposition and degradation can be achieved.

The formulations of above prior arts involve complex process and/or specialized excipients in solving the compatibility and/or stability concerns involved in the preparation of fixed dose combination of alogliptin and metformin.

Thus, there is a need to develop a simple and convenient process of achieving stability of alogliptin while it is present in a fixed dose combination with metformin.

Hence, the present invention is provides a stable fixed dose combination formulation comprising alogliptin and metformin.

### SUMMARY OF THE INVENTION

The present invention is relates to a pharmaceutical composition comprising alogliptin, metformin and one or more pharmaceutical excipients, wherein alogliptin, metformin, optionally pharmaceutical excipients, are present as a single granulated mass.

### DETAILED DESCRIPTION OF THE INVENTION

The present invention is relates to a pharmaceutical composition comprising alogliptin; metformin; as well as one or more pharmaceutical excipients.

In another embodiment the invention is relates to a pharmaceutical composition comprising alogliptin, metformin and one or more pharmaceutical excipients, wherein alogliptin, metformin, optionally pharmaceutical excipients, are present as a single granulated mass.

The term ‘single granulated mass’ comprises alogliptin, metformin and/or pharmaceutical excipients are granulated together.

The pharmaceutical excipients which used herein may be selected from the group consisting of one or more fillers, one or more binders or diluents, one or more lubricants, one or more disintegrants, and one or more glidants, one or more film-coating agents, one or more pigments, and the like.

The fillers can be selected from the group consisting of microcrystalline cellulose, lactose, D-mannitol, corn starch and/or pregelatinized starch etc and the like; the disintegrant can be selected from the group consisting of croscarmellose sodium, sodium starch glycolate, and the like.

The binder can be selected from the group consisting of Povidone, copovidone, hydroxypropyl methylcellulose (HPMC), hydroxypropyl cellulose (HPC) and the like. The, lubricant can be magnesium stearate and the like, and the glidant can be colloidal anhydrous silica and the like.

The pharmaceutical excipients used within this invention are conventional materials such as D-mannitol, pregelatinized starch and microcrystalline cellulose or silicified Microcrystalline cellulose as a filler, povidone as a binder, croscarmellose sodium as a disintegrant, magnesium stearate as a lubricant, colloidal anhydrous silica as a glidant, hypromellose (HPMC) as a film-coating agent, titanium dioxide, iron oxides as pigments, and talc, etc.

A typical composition according to the present invention comprises the binder povidone (also known as polyvinyl pyrrolidone or Kollidone 30 LP).

Further, a typical composition according to the present invention comprises the filler mannitol, the binder povidone, the lubricant magnesium stearate, and the glidant colloidal anhydrous silica.

A pharmaceutical composition according to an embodiment of the present invention is intended for the treatment of diabetes and/or to achieve glycaemic control in a type 1 or type 2 diabetes mellitus patient and comprises a fixed dose combination formulation as described herein together with suitable pharmaceutical excipients.

Thus, in particular, the present invention is directed to a pharmaceutical composition comprising alogliptin benzoate, metformin hydrochloride and one or more pharmaceutical excipients, particularly one or more fillers, one or more binders, one or more glidants, and/or one or more lubricants.

In more particular, the present invention is directed to a pharmaceutical composition comprising alogliptin benzoate, metformin hydrochloride, croscarmellose as disintegrant and one or more further pharmaceutical excipients.

Typical pharmaceutical compositions of this invention may comprise alogliptin portion 0.1-10% (e.g. more specifically, 2.5% by weight of total tablet core of uncoated tablet).

Typical pharmaceutical compositions of this invention may comprise one or more of the following amounts (% by weight of total coated tablet mass):

TABLE 1

| S. | | % of ingredient by |
|---|---|---|
| No | Ingredients | weight of core tablet |
| 1 | Alogliptin (as benzoate salt) | 0.1-10% |
| 2 | Metformin Hydrochloride | 40-85% |
| 3 | Filler | 0-50% |
| | (e.g. D-mannitol, Microcrystalline cellulose, | |
| | silicified Microcrystalline cellulose | |
| | Pregelatinized starch) | |
| 4 | Disintegrant | 0-20% |
| | (e.g. Croscarmellose sodium, Sodium starch | |
| | glycolate,) | |
| 5 | Binder | 0.1-10% |
| | (e.g. Povidone, Copovidone, HPMC, HPC) | |
| 6 | Lubricant | 0.1-10% |
| | (e.g. Magnesium stearate) | |
| 7 | Glidant | 0.1-5% |
| | (e.g. Colloidal silica anhydrous) | |

Further details about the fixed dose combination formulations of this invention, e.g. the ingredients, ratio of ingredients (such as e.g. ratio of alogliptin, metformin hydrochloride, and/or excipients), particularly with respect to special dosage forms (tablets) used within this invention as well as their preparation, become apparent to the skilled person from the disclosure hereinbefore and hereinafter (including by way of example the following examples as well as the claims).

In an embodiment, alogliptin in the context of the present invention is any form of base and/or pharmaceutically acceptable salt.

In a second embodiment, alogliptin in the context of the present invention is a crystalline benzoate salt.

According to this invention it is to be understood that the definition of alogliptin also comprise its pharmaceutically acceptable salts as well as hydrates, solvates and polymorphic forms thereof.

The dosage required when administered orally is 0.5 mg to 100 mg, preferably 2.5 mg to 50 mg or 0.5 mg to 30 mg, more preferably 12.5 mg to 25 mg, in each case 1 to 4 times a day.

A dosage form prepared with a pharmaceutical composition comprising alogliptin contains the active ingredient in a dosage range of 5-50 mg, in particular 12.5 to 25 mg. Thus, particular dosage strengths of alogliptin for inclusion into fixed dose combination pharmaceutical compositions of the present invention is12.5 or 25 mg.

Metformin is usually given in doses varying from about 250 mg to 3000 mg, particularly from 500 mg to 2000 mg up to 2500 mg per day using various dosage regimens.

A dosage range of the metformin is usually from 100 mg to 500 mg or 200 mg to 850 mg (1-3 times a day), or from 300 mg to 1000 mg once or twice a day. The unit dosage strengths of the metformin hydrochloride for use in the present invention may be from 100 mg to 2000 mg or from 250 mg to 2000 mg, preferably from 250 mg to 1000 mg. Particular dosage strengths may be 250, 500, 625, 750, 850 and 1000 mg of metformin hydrochloride. These unit dosage strengths of metformin hydrochloride represent the dosage strengths approved in the US for marketing to treat type 2 diabetes. More particular unit dosage strengths of metformin hydrochloride for incorporation into the fixed dose combination pharmaceutical compositions of the present invention are 500, 850 and 1000 mg of metformin hydrochloride.

The amount of the alogliptin and metformin in the pharmaceutical composition according to this invention correspond to the respective dosage ranges as provided hereinbefore. For example, a pharmaceutical composition comprises alogliptin in an amount of 12.5 mg to 25 mg (namely 12.5 mg or 25 mg) and of metformin hydrochloride in an amount of 250 mg to 1000 mg (namely 250, 500, so 625, 750, 850 or 1000 mg).

In another aspect, the further embodiment of the present invention provides a process for preparation of compositions, formulations, blends or dosage forms of this invention, such as e.g. by using methods known to one skilled in the art and/or in a manner as described herein, for example they may be obtained by processes comprising using (e.g. mixing, combining, blending and/or composing) the components and/or ingredients, or pre-mixtures thereof, mentioned hereinbefore and hereinafter, as well as the present invention further provides compositions, formulations, blends or dosage forms obtainable by these methods or processes and/or obtainable from the components, ingredients, pre-mixtures and/or mixtures mentioned hereinbefore and hereinafter.

In a further aspect of the present invention, the present invention provides a stable pharmaceutical composition, formulation, blend or dosage form of this invention which is substantially free of or only marginally comprises impurities and/or degradation products; that means, for example, that the composition, formulation, blend or dosage from includes about <5%, or about <4%, or about <3%, or less than about 2%, preferably less than about 1%, more preferably less than about 0.5%, even more preferably less than about 0.2% of any individual or total impurity or degradation product(s) by total weight. The content and/or degradation can be determined by well-known analytical methods, for example using HPLC methods.

Dosage Forms for the FDC Formulations of this Invention:

A typical mono-layer tablet of this invention comprises alogliptin, metformin hydrochloride, one or more fillers (such as e.g. mannitol), one or more binders (such as e.g. povidone or copovidone), one or more glidants (such as e.g. colloidal anhydrous silica) and one or more lubricants (such as e.g. magnesium stearate).

In a preferred embodiment of the present invention, the present invention is directed to a stable oral solid pharmaceutical composition, preferably a tablet, particularly a mono-layer tablet comprising or made from Alogliptin (particularly alogliptin benzoate e.g. in an amount of 12.5 mg), Metformin (particularly metformin hydrochloride, e.g. in an amount of 500 mg or 1000 mg), and one or more pharmaceutical excipients, particularly one or more fillers (e.g. mannitol, microcrystalline cellulose, silicified Microcrystalline cellulose, pregelatinized starch), one or more disintegrants (e.g. croscarmellose sodium), one or more binders (e.g. povidone), one or more glidants (e.g. colloidal anhydrous silica) and/or one or more lubricants (e.g. magnesium stearate), as well as, optionally, a film coat e.g. comprising one or more film-coating agents (e.g. hypromellose), (one or more pigments (e.g. titanium dioxide, iron oxide red and/or iron oxide yellow) and/or one or more glidants (e.g. talc).

A process for preparation of tablet of this invention comprises compression of one or more final blends in form of granules. Granules of the (final) blend(s) according to this invention may be prepared by methods well-known to one skilled in the art (e.g. high shear wet granulation or fluid bed granulation).

Granules according to this invention as well as details of granulation processes (including their separate steps) for the preparation of granules of this invention is described by way of example in the following examples.

An illustrative granulation process for the preparation of granules comprising the mono-layer composition comprises
i.) dissolving or dispersing a binder in a solvent or mixture of solvents such as isopropyl alcohol at ambient temperature to produce a granulation liquid;
ii.) blending alogliptin benzoate, metformin HCl, fillers, disintegrants, dry binder in a suitable mixer (e.g. high shear mixer granulator or fluid bed granulator) to produce a pre-mix;
iii.) spraying the granulation-liquid into the pre-mix and granulating the mixture for example in a fluid-bed granulator or high shear mixer granulator, preferably under dry condition;
iv.) drying the granulate, e.g. at about 55-60° C. inlet air temperature until the desired loss on drying value in the range of 1-2% is obtained;
v.) milling the dried granulate for example by sieving through a sieve with a mesh size of 0.5 to 2.0 mm;
vi.) blending the sieved granulate and preferably sieved extra-granular disintegrant), glidant in a suitable blender;
vii.) adding preferably sieved lubricant to the granulate for final blending for example in the conta blender.

Pharmaceutical immediate release dosage forms of this invention preferably have dissolution properties such that after 45 minutes for each of the active ingredients at least 75%, even more preferably at least 90% by weight of the respective active ingredient is dissolved.

In a particular embodiment, after 30 minutes for each of the active ingredients especially of the tablet according to this invention (including tablet core and film-coated tablet) at least 70-75% (preferably at least 80%) by weight of the respective active ingredient is dissolved. In a further embodiment, after 15 minutes for each of the active ingredients especially of the mono-layer tablet according to this invention (including tablet core and film-coated tablet) at least 55-60% by weight of the respective active ingredient is dissolved. The dissolution properties can be determined in standard dissolution tests, e.g. according to standard pharmacopoeias (e.g. using paddle method with agitation speed of 50 rpm).

Further embodiments, features and advantages of the present invention may become apparent from the following examples. The following examples serve to illustrate, by way of example, the principles of the invention without restricting it.

### Examples

### Film Coated Tablets

The composition of film coated tablets for alogliptin benzoate+metformin HCl fixed dose combination is shown in Table 2.

### Composition 1

TABLE 2

| | Strength | | | |
|---|---|---|---|---|
| | 12.5/1000 mg | | 12.5/500 mg | |
| | mg/ | % compo- | mg/ | % compo- |
| Ingredients | tablet | sition | tablet | sition |

Intragranular

| | | | | |
|---|---|---|---|---|
| Alogliptin Benzoate | 17 | 1.27 | 17 | 2.34 |
| Metformin Hydrochloride | 1000.00 | 74.63 | 500.00 | 69.44 |
| Mannitol | 69.00 | 5.15 | 50.00 | 7.08 |
| Microcrystalline Cellulose | 73.00 | 5.45 | 52.00 | 7.37 |
| Croscarmellose sodium | 45.00 | 3.38 | 23.50 | 3.34 |
| Pregelatininized starch | 20.00 | 1.49 | 10.50 | 1.49 |
| Povidone | 28.00 | 2.09 | 12.00 | 1.70 |

Granulating Fluid

| | | | | |
|---|---|---|---|---|
| Povidone | 12.00 | 0.90 | 5.50 | 0.78 |
| Isopropyl Alcohol | Qs | Qs | Qs | Qs |

Extragranular

| | | | | |
|---|---|---|---|---|
| Croscarmellose sodium | 20.00 | 1.50 | 10.50 | 1.48 |
| Colloidal Silicon Dioxide | 5.00 | 0.37 | 3.00 | 0.42 |
| Magnesium Stearate | 11.00 | 0.82 | 6.00 | 0.85 |
| Core tablet weight | 1300.00 | 97.75 | 690.00 | 97.73 |

Film coating

| | | | | |
|---|---|---|---|---|
| Opadry yellow | 30.00 | 2.25 | 16.00 | 2.27 |
| Coated tablet weight | 1330.00 | 100.00 | 716.00 | 100.00 |

### Manufacturing Procedure of Film Coated Tablets:

### 1) Dispensing:

Raw material dispensing: Dispense all raw materials as per mentioned formula.

### 2) Sifting:

Sift alogliptin benzoate, metformin hydrochloride, microcrystalline cellulose, mannitol, pregelatinized starch and croscarmellose sodium and povidone through #30 mesh.

### 3) Preparation of Granulating Fluid:

Weigh required quantity of isopropyl alcohol and keep under stirring. Add povidone into the vortex of isopropyl alcohol. Stir until a clear homogeneous solution is obtained.

### 4) Granulation:

Load premix blend of Step 2 in rapid mixer granulator. Mix the blend for 10 minutes at impeller slow and chopper off. Granulate blend using binder solution of step 3 at impeller slow, chopper slow.

### 5) Drying:

Dry the wet granules of step 4 in fluid bed dryer at 55-60° C. until desired LOD achieved around 2% at 105° C. in Halogen moisture analyzer. Sift the dried granules of step 5 through multi mill with knives forward in slow speed.

### 6) Sifting of Extra-Granular Ingredients:

Sift croscarmellose sodium, colloidal silicon dioxide and magnesium stearate through #40 & #60 mesh respectively. Mix Step No 5 with croscarmellose sodium in conta blender for 15 minutes (Blender speed: 16 RPM).

### 7) Lubrication:

Mix step No. 6 with magnesium stearate and lubricate for 5 minutes in conta blender.

### 8) Compression:

Compress the lubricated blend of step 7 using appropriate punches.

The tablet cores may be film-coated by an aqueous film-coating suspension, containing hypromellose as film-forming agent, suitable, talc as glidant and the pigments yellow iron oxide and/or red iron oxide and titanium dioxide etc.

### Composition 2

TABLE 3

| | Strength | | | |
|---|---|---|---|---|
| | 12.5/1000 mg | | 12.5/500 mg | |
| Ingredients | mg/tablet | % composition | Ingredients | mg/tablet |

Intragranular

| | | | | |
|---|---|---|---|---|
| Alogliptin | 17.00 | 1.25 | 17.00 | 2.43 |
| benzoate | | | | |
| Metformin | 1000.00 | 73.53 | 500.00 | 71.53 |
| Hydrochloride | | | | |
| Pregelatinized | 15.00 | 1.10 | 7.70 | 1.10 |
| starch | | | | |
| Mannitol | 65.00 | 4.78 | 33.40 | 4.78 |
| Microcrystalline | 66.00 | 4.85 | 39.52 | 5.65 |
| Cellulose | | | | |
| Croscarmellose | 45.00 | 3.31 | 23.14 | 3.31 |
| sodium | | | | |
| Povidone | 28.00 | 2.06 | 14.40 | 2.06 |
| (plasdone k-90) | | | | |
| Povidone | 30.00 | 2.21 | 15.45 | 2.21 |
| (Kollidone30LP) | | | | |
| IPA | q.s | — | q.s | — |

Extra Granuler

| | | | | |
|---|---|---|---|---|
| Croscarmellose | 32.00 | 2.35 | 16.44 | 2.35 |
| sodium | | | | |
| Silified | 34.00 | 2.50 | 17.50 | 2.50 |
| microcrystalline | | | | |
| cellulose | | | | |
| Magnesium | 8.00 | 0.59 | 4.15 | 0.59 |
| stearate | | | | |
| Core tablet | 1340.00 | 98.53 | 688.70 | 98.51 |
| weight | | | | |
| Film coating | | | — | — |
| Coated tablet | 1360.00 | 100.00 | 699.00 | 100.00 |
| weight | | | | |

### Manufacturing Procedure of Film Coated Tablets:

### 1. Dispensing:

Raw material dispensing: Dispense all raw materials as per mentioned formula.

### 2. Sifting:

Sift Alogliptin benzoate, Metformin hydrochloride, microcrystalline cellulose, Mannitol, Pregelatinized Starch (Lycatab C), Croscarmellose Sodium (AC-DI-SOL) and Povidone K-90 (Plasdone K-90 D) through #30 mesh.

### 3. Preparation of Granulating Fluid:

Weigh required quantity of Isopropyl Alcohol. Add povidone (Kollidone30 LP) under the vortex of Isopropyl alcohol. Continue stirring to get clear homogeneous solution.

### 4. Granulation:

Load premix blend of Step 2 in rapid mixer granulator. Mix the blend for 10 minutes at impeller slow and chopper off. Granulate blend using binder solution of step 3 at impeller slow, chopper slow.

### 5. Drying:

Dry the wet granules of step 4 in fluid bed dryer at 55-60° C. until desired LOD achieved around 2% at 105° C. in Halogen moisture analyzer. Sift the dried granules of step 5 through multi mill with knives forward in slow speed.

### 6. Sifting of Extra-Granulars:

Sift Cross-carmellose sodium (AC-DI-SOL) and Silicified microcrystalline cellulose (Prosolv SMCC-90) and magnesium stearate through #40 & #60 mesh respectively. Mix Step No 5 with Cross-carmellose sodium (AC-DI-SOL) and Silicified microcrystalline cellulose (Prosolv SMCC-90) in conta blender for 15 minutes (Blender speed: 16 RPM).

### 7. Lubrication:

Mix step No. 6 with magnesium stearate and lubricate for 5 minutes in conta blender (Blender Speed: 16 RPM).

### 8. Compression:

Compress the lubricated blend of step 7 using appropriate punches.

### 9. Coating:

Coat the uncoated tablets of step 8 using coating machine.

### Composition 3

TABLE 4

| | Strength | | | |
|---|---|---|---|---|
| | 12.5/1000 mg | | 12.5/500 mg | |
| | mg/ | % compo- | mg/ | % compo- |
| Ingredients | tablet | sition | tablet | sition |

Intragranular

| | | | | |
|---|---|---|---|---|
| Alogliptin benzoate | 17.17 | 1.27 | 17.17 | 2.35 |
| Metformin Hydrochloride | 1000.00 | 74.07 | 500.00 | 68.49 |
| Mannitol | 93.83 | 6.95 | 69.83 | 9.57 |
| Microcrystalline Cellulose | 72.00 | 5.33 | 47.00 | 6.44 |
| cross carmellose sodium | 45.00 | 3.33 | 25.00 | 3.42 |
| Povidone | 23.00 | 1.70 | 13.00 | 1.78 |

Granulating Fluid

| | | | | |
|---|---|---|---|---|
| Povidone | 27.00 | 2.00 | 15.00 | 2.05 |
| Isopropyl Alcohol | Qs | Qs | Qs | Qs |

Extra granular

| | | | | |
|---|---|---|---|---|
| cross carmellose sodium | 20.00 | 1.58 | 12.00 | 1.64 |
| Magnesium Stearate | 17.00 | 1.26 | 11.00 | 1.51 |
| Core tablet weight | 1315.00 | 97.41 | 710.00 | 97.26 |

Film coating

| | | | | |
|---|---|---|---|---|
| Opadry yellow | 35.00 | 2.59 | 20.00 | 2.74 |
| Coated tablet weight | 1350.00 | 100.00 | 730.00 | 100.00 |

### Manufacturing Procedure of Film Coated Tablets:

### 1. Dispensing:

Raw material dispensing: Dispense all raw materials as per mentioned formula.

### 2. Sifting:

Sift Alogliptin benzoate, Metformin hydrochloride, microcrystalline cellulose, Mannitol and cross carmellose sodium and povidone through #30 mesh.

### 3. Preparation of Granulating Fluid:

Weigh required quantity of Isopropyl Alcohol. Add povidone under the vortex of Isopropyl alcohol. Continue stirring to get clear homogeneous solution.

### 4. Granulation:

Load premix blend of Step 2 in rapid mixer granulator. Mix the blend for 10 minutes at impeller slow and chopper off. Granulate blend using binder solution of step 3 at impeller slow, chopper slow.

### 5. Drying:

Dry the wet granules of step 4 in fluid bed dryer at 55-60° C. until desired LOD achieved around 2% at 105° C. in Halogen moisture analyzer. Sift the dried granules of step 5 through multi mill with knives forward in slow speed.

### 6. Sifting of Extra-Granulars:

Sift cross carmellose sodium and magnesium stearate through #40 & #60 mesh respectively. Mix Step No 5 with cross carmellose sodium in conta blender for 15 minutes (Blender speed: 16 RPM).

### 7. Lubrication:

Mix step No. 6 with magnesium stearate and lubricate for 5 minutes in conta blender (Blender Speed: 16 RPM).
   8. Compression: Compress the lubricated blend of step 7 using appropriate punches.

### 9. Coating:

Coat the uncoated tablets of step 8 using coating machine.

### Stability Data:

Stability data of Alogliptin benzoate+Metformin HCl fixed dose combination film coated tablets is shown in the following tables (over 2 months and 3 months):

TABLE 5

| | | | | |
|---|---|---|---|---|
| | Specification | | 2 M | 3 M |
| Condition | as per ICH | Initial | 40° C./75% RH | 40° C./75% RH |

Related Substance for Alogliptin

| | | | | |
|---|---|---|---|---|
| Impurity I | NMT 0.5% | 0.02 | 0.018 | 0.006 |
| Impurity II | NMT 0.5% | 0.011 | 0.013 | 0.016 |
| Impurity III | NMT 0.5% | ND | 0.012 | ND |
| Impurity IV | NMT 0.5% | ND | ND | ND |
| Impurity V | NMT 0.5% | ND | ND | ND |
| Highest | 0.20% | 0.024 | 0.079 | 0.121 |
| Unknown | | | | |
| impurity | | | | |
| Total | | 0.082 | 0.208 | 0.270 |

RS for meftormin HCl

| | | | | |
|---|---|---|---|---|
| Related comp. A | 0.20% | 0.002 | 0.003 | 0.003 |
| Related comp. B | 0.20% | ND | ND | ND |
| Related comp. C | 0.20% | 0.001 | ND | ND |
| Melamine | 0.20% | 0.001 | ND | 0.003 |
| Highest | 0.20% | 0.013 | ND | 0.006 |
| Unknown | | | | |
| impurity | | | | |
| Total impurity | | 0.028 | 0.026 | 0.026 |
| Assay (%) | Between | 101.8% | 101.5% | 102.3% |
| Alogliptin | 90%-110% | | | |
| Assay (%) | Between | 101.2% | 101.4% | 99.9% |
| Metformin HCl | 90%-110% | | | |
| % Water by KF | NMT 5% | 1.63% | 1.75% | 1.92% |

Related Substance for Alogliptin

| | |
|---|---|
| Impurity I | 2-({6-[(3R)-1-{3-[(O-cyanophenyl)methyl]-1-methyl-2,6-dioxo-1,3- |
| | dihydropyrimidin-4-yl}-3-piperidylamino]-3-methyl-2,4-dioxo-1,3- |
| | dihyropyrimidin-1-yl}methyl)benzonitrile (Dimer impurity) |
| Impurity II* | 2-({3-methyl-2,4-dioxo-6-[(3R)-piperidin-3ylamino]-3,4- |
| | dihydropyrimidin-1(2H)-yl}methyl)benzonitirile (Regio isomer) |
| Impurity III | 2-({6-[(3R)-3-aminopiperidin-1-yl]-2,4-dioxo-3,4-dihyropyrimidin- |
| | 1(2H)-yl}methyl)benzonitrile monobenzoate (N-desmethyl |
| | alogliptin) |
| Impurity IV | O-({4-[(3R)-3-Aminocyclohyxyl]-3-[(o-cyanophenyl)methyl]-2,6- |
| | dioxo-1,3-dihydropyrimidin-1-yl}methyl)benzonitirile benzoate |
| | (dialkyl impurity) |
| Impurity V* | 2-({6-[(3R)-3-aminopiperidin-1-yl]-3-methyl-2,4-dioxo-3,4- |
| | dihydropyrimidin-1(2H)-yl}methyl)benzamide |
| S-Isomer | 2-[[6-[(3R)-3-amino-1-piperidinyl]-3,4-dihydro-3-methyl-2,4-dioxo- |
| | 1(2H)pyrimidinyl]methyl]-benzonitrile monobenzoate |

*Impurity will be exist as a benzoate salt in API

TABLE 6

| | | | |
|---|---|---|---|
| Time | | 2 M | 3 M |
| points | Initial | 40° C./75% RH | 40° C./75% RH |

Alogliptin Dissolution Condition:
900 ml, 0.01 mol/lit hydrochloric acid; USP-II @50 rpm

| | | | | |
|---|---|---|---|---|
| 5 min | NLT 85% in 15 | 85 | 87 | 90 |
| 10 min | Minutes | 97 | 100 | 101 |
| 15 min | | 99 | 102 | 101 |
| 20 min | | 98 | 102 | 102 |
| 30 min | | 98 | 102 | 103 |

Metformin HCl Dissolution Condition:
900 ml, 0.01 mol/lit hydrochloric acid; USP-II @50 rpm

| | | | | |
|---|---|---|---|---|
| 5 min | NLT 85% in 15 | 85 | 85 | 89 |
| 10 min | Minutes | 97 | 98 | 101 |
| 15 min | | 99 | 100 | 101 |
| 20 min | | 99 | 101 | 102 |
| 30 min | | 98 | 102 | 102 |

1. A pharmaceutical stable composition comprising alogliptin, metformin and one or more pharmaceutical excipients, wherein alogliptin, metformin, optionally pharmaceutical excipients, are present as a single granulated mass.

2. The composition as claimed in claim 1, wherein the alogliptin is present in a dosage range from about 5 mg to about 50 mg.

3. The composition as claimed in claim 1, wherein said metformin is metformin hydrochloride and is present in a dosage range from about 100 mg to about 1500 mg.

4. The composition as claimed in claim 1, wherein the excipients are selected from the group consisting of one or more fillers; a disintegrant, a binder; a lubricant and a glidant.

5. The composition as claimed in claim 1, wherein the binder is povidone.

6. The composition as claimed in claim 1, wherein the in the said composition is a granules, capsules, tablet and a film coated tablet.

7. A process for preparing a pharmaceutical composition as claimed in claim 1 comprising incorporating the active ingredients in one or more pharmaceutical excipients selected from mannitol, microcrystalline cellulose, pregelatinized starch, cross carmellose sodium, povidone, silicified microcrystalline cellulose and magnesium stearate

8. A process for preparing a pharmaceutical composition as claimed in claim 1 comprising wet granulation of alogliptin benzoate along with metformin hydrochloride and other optional pharmaceutically acceptable excipients.
